(19) United States
(12) Patent Application Publication
(10) Pub. No.: US 2024/0285902 A1
(43) Pub. Date: Aug. 29, 2024

(54) MULTIPLE WIRE CATHETER BRAID WITH WIRE COUNT GRADIENT

(71) Applicants: DePuy Synthes Products, Inc., Raynham, MA (US); Neuravi Limited, Galway (IE)

(72) Inventors: Pedro PEDROSO, Raynham, MA (US); Joshua LIEBOWITZ, Raynham, MA (US); Karl KEATING, Galway (IE)

(73) Assignees: DePuy Synthes Products, Inc., Raynham, MA (US); Neuravi Limited, Galway (IE)

(21) Appl. No.: 18/113,160

(22) Filed: Feb. 23, 2023

(51) Int. Cl. A61M 25/00; A61M 25/01

(52) U.S. Cl. CPC A61M 25/0053; A61M 25/0147

(57) ABSTRACT

The disclosed technology can include a catheter braid that can comprise a first region, which can comprise a plurality of wire segments arranged in a first weave pattern, and a second region, which can have a plurality of wire segments arranged in a second weave pattern. The first weave pattern can transition to the second weave pattern at a first transitional point, which can be disposed at an intersection of the first region and the second region. The first transitional point can be where at least one wire of the plurality of wires of the first region can be removed from the second region. The catheter braid can comprise a uniform cross section area across a majority of the first region and a majority of the second region.

### FIELD OF INVENTION

This invention generally relates to devices and methods for accessing blood vessels during intravascular medical treatments. More specifically, the present disclosure relates to a catheter with a braided support structure.

### BACKGROUND

Catheters serve a broad range of functions in intravascular medical treatments. Catheters typically include a thin tube manufactured from medical grade materials that can be inserted into a body and can be used to deliver drugs or other devices, perform surgical procedures, remove blockages from vessels, and a variety of other purposes. By modifying the material or adjusting the way a catheter is manufactured, it is possible to tailor different sections of the catheter for particular applications.

There have been a plethora of designs and methods proposed for navigating a catheter to a specified point. Current catheter designs typically incorporate several polymeric durometers, utilized to transition the stiffness of the catheter from very stiff to less stiff materials towards the distal tip of the catheter. The functional efficacy of catheter designs is evaluated based on their ability to track through specific anatomy, flexibility, and track through said anatomy with as little frictional force as possible, push efficiency. Although the aforementioned catheter design approach transitions stiffness of materials along the length of the catheter to provide greater degrees of flexibility, column strength is sacrificed due to plastics typically being weaker than metals. These design tradeoffs considered with current catheter designs present considerable challenges to practitioners implementing them within highly tortuous anatomies. Furthermore, lack of column strength can result in an increased likelihood of a catheter device buckling which decreases the functional effectiveness and overall procedural success.

Thus, there is a need for systems, devices and methods that can provide improvements to catheter designs, specifically providing improvements in push efficiency and flexibility while maintaining column strength when utilized within anatomical environments with high degrees of tortuosity.

### SUMMARY

An exemplary embodiment of the present disclosure provides a catheter braid that can includes a first region, which can include a plurality of wire segments arranged in a first weave pattern that can have a first picks per inch, and a second region, which can have a plurality of wire segments arranged in a second weave pattern that can have a second picks per inch. The second picks per inch can be less than the first picks per inch and the second region can be distal of the first region. The first weave pattern can transition to the second weave pattern at a first transitional point, which can be disposed at an intersection of the first region and the second region. The first transitional point can be where at least one wire of the plurality of wires of the first region can be removed from the second region, which can reduce the first picks per inch to the second picks per inch. The catheter braid can have a uniform cross section area across a majority of the first region and a majority of the second region.

In any of the embodiments disclosed herein, the first weave pattern can be a 4×4 arrangement of the plurality of wire segments of the first region.

In any of the embodiments disclosed herein, the second weave pattern can be a 4×3 arrangement of the wire segments of the second region.

In any of the embodiments disclosed herein, the catheter braid can further include a third region, which can include a plurality of wire segments arranged in a third weave pattern that can have a third picks per inch. The third picks per inch can be less than the second picks per inch and the third region can be distal of the second region. The second weave pattern can transition to the third weave pattern at a second transitional point, which can be disposed at an intersection of the second region and the third region. The second transitional point can be where at least one wire of the plurality of wires of the second region is removed from the third region, which can reduce the second picks per inch to the third picks per inch.

In any of the embodiments disclosed herein, the third weave pattern can be a 3×3 arrangement of the wire segments of the third region.

In any of the embodiments disclosed herein, the catheter braid can further include a fourth region, which can include a plurality of wire segments arranged in a fourth weave pattern that can have a fourth picks per inch. The fourth picks per inch can be less than the third picks per inch and the fourth region can be distal of the third region. The third weave pattern can transition to the fourth weave pattern at a third transitional point, which can be disposed at an intersection of the third region and the fourth region. The third transitional point can be where at least one wire of the plurality of wires of the third region can be removed from the fourth region, which can reduce the third picks per inch to the fourth picks per inch.

In any of the embodiments disclosed herein, the fourth weave pattern can be a 2×2 arrangement of the wire segments of the fourth region.

In any of the embodiments disclosed herein, the catheter braid can further include a fifth region, which can include a plurality of wire segments arranged in a fifth weave pattern that can have a fifth picks per inch. The fifth picks per inch can be less than the fourth picks per inch and the fifth region can be distal of the fourth region. The fourth weave pattern can transition to the fifth weave pattern at a fourth transitional point, which can be disposed at an intersection of the fourth region and the fifth region. The fourth transitional point can be where at least one wire of the plurality of wires of the fourth region can be removed from the fifth region, which can reduce the fourth picks per inch to the fifth picks per inch.

In any of the embodiments disclosed herein, the fifth weave pattern can be a 1×1 arrangement of the wire segments of the fifth region.

In any of the embodiments disclosed herein, the first weave pattern can have a braid angle approximately equal to a braid angle of the second weave pattern.

Another embodiment of the present disclosure provides a catheter braid that can include a first region, which can include a first plurality of wire segments, a second region, which can include a second plurality of wire segments that can be fewer than the first plurality of wire segments, and a third region, which can include a third plurality of wire segments that can be more than the second plurality of wire segments.

In any of the embodiments disclosed herein, at least a portion of the third plurality of wire segments can include distal hoops.

In any of the embodiments disclosed herein, the third region can be configured to expand to an outer diameter that can be greater than an outer diameter of the second region and an outer diameter of the first region when unconstrained.

In any of the embodiments disclosed herein, the second plurality of wire segments can consist of wire segments wound in a single direction.

In any of the embodiments disclosed herein, the terminations of wires between the first, second, and third regions can be secured through a laser weld or adhesive bond.

Another embodiment of the present disclosure provides a catheter braid that includes a first region, which can include a first plurality of wire segments arranged in a first weave pattern, a second region, which can include a second plurality of wire segments arranged in a second weave pattern. The second plurality of wire segments can include fewer wire segments than the first plurality of wire segments. The first region can have a circumference approximately equal to a circumference of the second region.

In any of the embodiments disclosed herein, the first weave pattern can include four distinct wire segments of the first plurality of wire segments that can be traversed over four distinct wire segments of the first plurality of wire segments.

In any of the embodiments disclosed herein, the second weave pattern can include four distinct wire segments of the second plurality of wire segments that can be traversed over three distinct wire segments of the second plurality of wire segments.

In any of the embodiments disclosed herein, a first portion of the first plurality of wire segments can terminate at a transition between the first portion and the second portion. The second plurality of wires can include wire segments extending from a second portion of the first plurality of wire segments.

In any of the embodiments disclosed herein, the catheter braid can further include a third region can include a third plurality of wire segments arranged in a third weave pattern, wherein the third plurality of wire segments can include fewer wire segments than the second plurality of wire segments. The first portion of the second plurality of wire segments can terminate at a transition between the second portion and the third portion. The third plurality of wire segments can include wire segments extending from a second portion of the second plurality of wire segments.

In any of the embodiments disclosed herein, the third weave pattern can include three distinct wire segments of the third plurality of wire segments that can be traversed over three distinct wire segments of the third plurality of wire segments.

In any of the embodiments disclosed herein, the catheter braid can further include fourth region can include a fourth plurality of wire segments that can be arranged in a fourth weave pattern, wherein the fourth plurality of wire segments can include fewer wire segments than the third plurality of wire segments. The first portion of the third plurality of wire segments can terminate at a transition between the third portion and fourth portion. The fourth plurality of wire segments can include wire segments that can extend from a second portion of the third plurality of wire segments.

In any of the embodiments disclosed herein, the fourth weave pattern can include two distinct wire segments of the fourth plurality of wire segments that can be traversed over two distinct wire segments of the fourth plurality of wire segments.

In any of the embodiments disclosed herein, the catheter braid can further include a fifth region that can include a fifth plurality of wire segments arranged in a fifth weave pattern, wherein the fifth plurality of wire segments can include fewer wire segments than the fourth plurality of wire segments. A first portion of the fourth plurality of wire segments can terminate at a transition between the fourth portion and the fifth portion. The fifth plurality of wire segments can include wire segments that can extend from a second portion of the fourth plurality of wire segments.

In any of the embodiments disclosed herein, the fifth weave pattern can include one wire segment of the fifth plurality of wire segments that can be traversed over one wire segment of the fifth plurality of wire segments.

In any of the embodiments disclosed herein, the first weave pattern can include a braid angle that can be approximately equal to a braid angle of the second weave pattern.

Another embodiment of the present disclosure provides a device that can include a tubular braid that can include a first region, which can include a plurality of strand segments arranged in a first weave pattern that can have a first picks per inch, and a second region, which can include a plurality of strand segments arranged in a second weave pattern having a second picks per inch that can be less than the first picks per inch. The second region can be distal of the first region. The first weave pattern can transition to the second weave pattern at a first transitional point that can be disposed at an intersection of the first region and the second region. The first transitional point can be where at least one strand of the plurality of strand segments of the first region can be removed from the second region, which can reduce the first picks per inch to the second picks per inch. The tubular braid can include a uniform circumference across the first region and the second region.

In any of the embodiments disclosed herein, the strand segments can include wire strands.

In any of the embodiments disclosed herein, the device can further include a catheter body supported by the tubular braid.

In any of the embodiments disclosed herein, the first weave pattern can have a braid angle that can be approximately equal to the braid angle of the second weave pattern.

Another embodiment of the present disclosure provides a method. The method can include arranging a first plurality of strand segments in a first weave pattern that can have a first picks per inch, terminating a first portion of the first plurality of strand segments at a first transition point, and arranging a second plurality of strand segments in second weave pattern that can have a picks per inch less than the first picks per inch. The second weave pattern can be distal of the first transition point such that the second plurality of wire segments can extend from a second portion of the first plurality of strand segments. The second weave pattern can have a circumference that can be approximately equal to the first weave pattern.

These and other aspects of the present disclosure are described in the Detailed Description below and the accompanying drawings. Other aspects and features of embodiments will become apparent to those skilled in the pertinent art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is an illustration of an example embodiment of a catheter braid, according to aspects of the present invention.

FIG. 2 is an illustration of an example catheter braid showing the first transitional point between the first region and second region, according to aspects of the present invention.

FIG. 3 is an illustration of an example catheter braid showing the second transitional point between the second region and the third region, according to aspects of the present invention.

FIG. 4 is an illustration of an example catheter braid showing the third transitional point between the third region and the fourth region, according to aspects of the present invention.

FIG. 5 is an illustration of a method flow chat describing a method of manufacture of an example catheter braid, according to aspects of the present invention.

FIGS. 6A and 6B are illustrations of an example embodiment of a catheter braid, according to aspects of the present invention.

FIGS. 6C. 6D, and 6E are cross-sectional illustrations of the catheter braid as indicated in FIG. 6A, according to aspects of the present invention.

FIGS. 7A and 7B are illustrations of an example embodiment of a catheter braid, according to aspects of the present invention.

FIGS. 7C, 7D, and 7E are cross-sectional illustrations of the catheter braid as indicated in FIG. 7A, according to aspects of the present invention.

FIG. 8A is an illustration of an example embodiment of a catheter braid, according to aspects of the present invention.

FIGS. 8B, 8C, 8D, and 8E are cross-sectional illustrations of the catheter braid as indicated in FIG. 8A, according to aspects of the present invention.

### DETAILED DESCRIPTION

The disclosed technology can include a catheter braid subdivided into regions with distinct weave patterns, wherein each of the regions can include a plurality of metal wire segments arranged into said weave patterns. The regions of the catheter braid can include varying numbers of metal wire segments over the length of the catheter braid to provide a relatively soft, flexible shaft at the distal portion, relatively stiff shaft at the proximal portion, and a graduated transition between the flexible and stiff shaft portions. To achieve the graduated transition between the flexible and stiff shaft portions, at least one metal wire segment of the plurality of metal wire segments, which can include each of the subdivided regions, can be removed at one or more transitional points along the length of the catheter braid. By gradually removing at least one metal wire segment of the plurality of metal wire segments at one or more transitional points along the length of the catheter braid, the likelihood of kinks can be reduced while push efficiency and column strength can be maintained.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, vasculature can be that of any “subject” or “patient” including of any human or animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, the terms “about” or “approximately” for any numerical values or ranges indicate a suitable dimensional tolerance that allows the part or collection of components to function for its intended purpose as described herein. More specifically, “about” or “approximately” may refer to the range of values±20% of the recited value, e.g. “about 90%” may refer to the range of values from 71% to 99%.

The figures illustrate a generally hollow or tubular structure according to the present invention. When used herein, the terms “tubular” and “tube” are to be construed broadly and are not limited to a structure that is a right cylinder or strictly circumferential in cross-section or of a uniform cross-section throughout its length. For example, the tubular structure or system is generally illustrated as a substantially right cylindrical structure. However, the tubular system may have a tapered or curved outer surface without departing from the scope of the present invention.

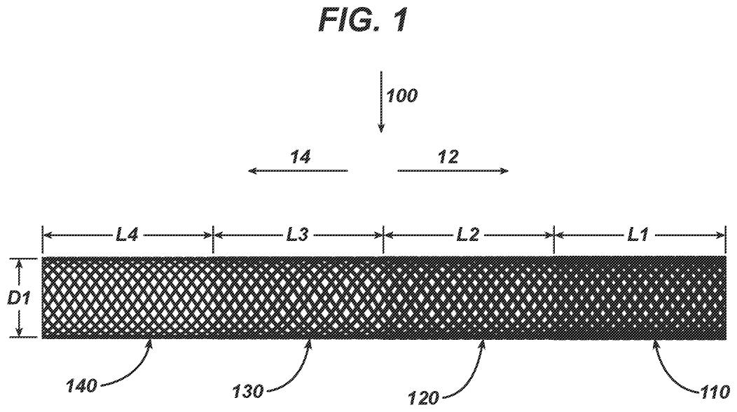

FIG. 1 illustrates an example catheter braid (100). The catheter braid (100) can have a diameter (D1) and can extend along a longitudinal axis (10) in the proximal direction (12) and the distal direction (14). The catheter braid (100) can be subdivided into multiple regions (110, 120, 130, 140) that can include distinct weave patterns for each of the multiple regions. The diameter (D1) of the braid (100) can be substantially constant throughout the regions (110, 120, 130, 140) such that each region has an approximately equal diameter. For a braid having a circular cross-section, a constant diameter (D1) means that the cross-sectional area of a lumen through the braid (100) is constant and a circumference of the braid (100) is constant. The braid can have alternative cross-sectional shapes and maintain a constant diameter (D1), cross-sectional area, or circumference through the regions (110, 120, 130).

The catheter braid (100) can include a first region (110), which can include a first weave pattern with a length (L1) along the longitudinal axis (10), a second region (120) which can include a second weave pattern with a length (L2) along the longitudinal axis (10), a third region (130) which can include a third weave pattern with a length (L3) along the longitudinal axis (10), and a fourth region (140), which can include a fourth weave pattern with a length (L4) along the longitudinal axis (10). The catheter braid (100) can also have a uniform cross section across a majority of the first region (110) and a majority of the second region (120). Each of the distinct regions of the catheter braid (100) can include a plurality of wire segments. The wire segments can be made of from one or a combination of stainless steel, cobalt chromium, molybdenum and niobium alloy, tungsten, nitinol, tantalum, platinum or any other hard metal alloy. The catheter braid (100) can have varying picks per inch along the length of each of the subdivided regions. For example, the first region (110) of the catheter braid (100) can have a first weave pattern that is denser than the second weave pattern of the second region (120); the second weave pattern can be denser than a third weave pattern of the third region (130); and the third weave pattern can be denser than a fourth weave pattern of the fourth region (140). Axial rigidity of a catheter supported by the braid (100) can therefore decrease distally due to decrease in density of the weave patterns of the braid (100) in the distal direction (14).

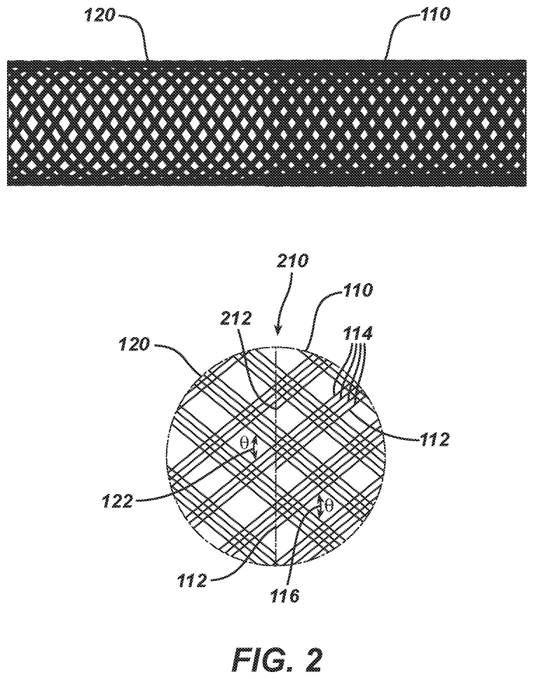

FIGS. 2 through 4 illustrate transitions between the regions (110, 120, 130, 140). Each of the distinct regions (110, 120, 130, 140) of the catheter braid (100) can transition at a transitional point that can be disposed between two distinct regions. The number and arrangement of wire segments illustrated in FIGS. 2 through 4 and described in relation to FIGS. 2 through 4 are presented for the sake of illustration, and numerous other numbers and arrangements of wire segments can be utilized as a variation on the presented examples. Such variations as understood by a person skilled in the pertinent art are within the scope of the present disclosure.

FIG. 2 illustrates a transition between the first region (110) and the second region (120). The first region (110) weave pattern can be arranged in a 4×4 arrangement of wire segments. The second region (120) weave pattern can be arranged in a 4×3 arrangement of wire segments. The first weave pattern of the first region (110) can transition to the second region (120) at a first transitional point (210) at the intersection of the first region (110) and the second region (120). The first transitional point (210) can be marked by the removal of at least one wire of the plurality of wires of the first region (110) from the second region (120). Resultantly, the removal of at least one wire of the plurality of wires of the first region (110) from the second region can lower the picks per inch of the first region (110) to the second picks per inch of the second region (120).

The first plurality of wire segments of the first region (110) can be arranged in a first weave pattern with a braid angle (116), and the second plurality of wire segments of the second region (120) can be arranged in a second weave pattern with a braid angle (122) approximately equal to the braid angle (116) of the first region (110). Therefore, the braid (100) can decrease in picks per inch while maintaining a constant braid angle (116, 122) from one region to the next. The first region (110) can have a circumference that can be approximately equal to a circumference of the second region (120).

The first region (110) weave pattern can include four distinct wire segments of the first plurality of wire segments that can be traversed over four distinct wire segments of the first plurality of wire segments. The second region (120) weave pattern can include four distinct wire segments of the second plurality of wire segments that can be traversed over three distinct wire segments of the second plurality of wire segments. A first portion (112) of the first plurality of wire segments can terminate (212) at the first transitional point (210). The second plurality of wire segments of the second region (120) can include wire segments that can extend from a second portion (114) of the first plurality of wire segments through the first transitional point (210).

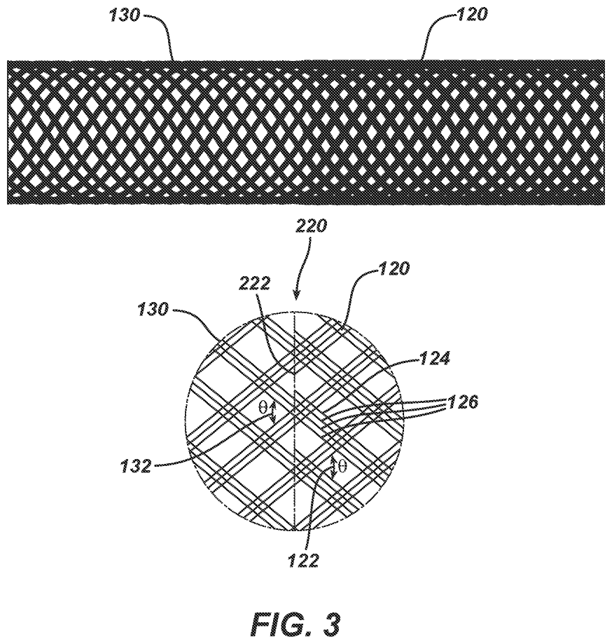

FIG. 3 is an illustration of the transition between the second region (120) and the third region (130) of the catheter braid (100). The third region (130) can include a plurality of wire segments that can be arranged in a third weave pattern, having a third picks per inch less than a second picks per inch of the second region, and can be disposed distal (14) of the second region (120). The second region (120) transitions to the third region at a second transitional point (220) that can be disposed at the intersection of the second region (120) and the third region (130). The second transitional point (220) can be where at least one wire of the plurality of wire segments of the second region (120) is removed from the third region (13), which reduces the second picks per inch of the second region (120) to the third picks per inch of the third region (130).

The third plurality of wire segments of the third region (130) can be arranged in a third weave pattern with a braid angle (132) approximately equal to the braid angle (122) of the second region (120) and approximately equal to the braid angle (116) of the first region (110). Therefore, the braid (100) can decrease in picks per inch while maintaining a constant braid angle (116, 122, 132) from one region to the next. The third region (130) can have a circumference that can be approximately equal to a circumference of the second region (120).

The third plurality of wire segments of the third region (130) can include fewer segments than the second plurality of wire segments of the second region (120). The third region (130) weave pattern can include three distinct wire segments of the third plurality of wire segments that can be traversed over three distinct wire segments of the third plurality of wire segments. A first portion (124) of the second plurality of wire segments can terminate (222) at the second transitional point (220). The second plurality of wire segments (126) of the second region can include wire segments that can extend through the second transitional point (220) into the third region (130).

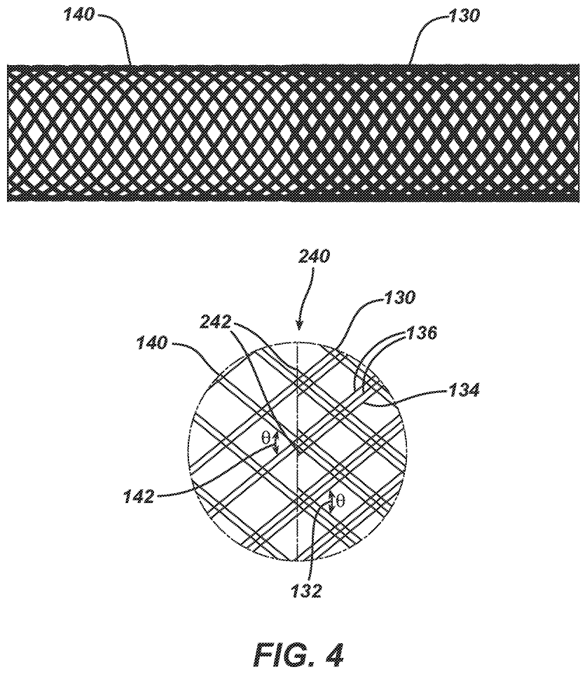

FIG. 4 is an illustration of the transition (240) between the third region (130) and the fourth region (140) of the catheter braid (100). The fourth region (140) can include a plurality of wire segments that can be arranged in a fourth weave pattern having a fourth picks per inch that can be less than the third picks per inch. The fourth region (140) can be distal (14) the third region (130). The fourth region (140) weave pattern can be a 2×2 arrangement of the wire segments. The third region (130) can transition to the fourth region (140) at a third transitional point (240) that can be disposed at an intersection of the third region (130) and the fourth region (140). The third transitional point (240) can be where at least one wire of the plurality of wire segments of the third region (130) is removed from the fourth region (140), which can reduce the third picks per inch to the fourth picks per inch.

The fourth region (140) can include a fourth plurality of wire segments that can be arranged in a fourth weave pattern. The fourth plurality of wire segments can include fewer segments than the third plurality of wire segments of the third region (130). The fourth region (140) weave pattern can include two distinct wire segments of the fourth plurality of wire segments that can be traversed over two distinct wire segments of the four plurality of wire segments. A first portion of the third plurality of wire segments (134) can terminate (242) at said third transitional point (240). The fourth plurality of wire segments of the fourth region (140) can include wire segments that can extend from a second portion of the second plurality of wire segments (136) through the third transitional point (240) to the fourth region (140).

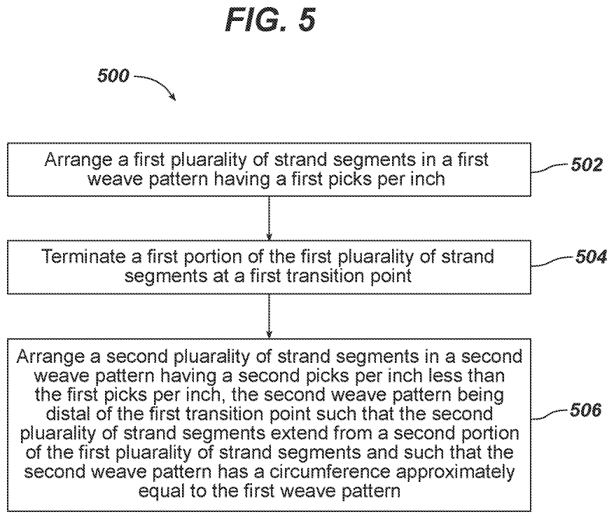

FIG. 5 illustrates a method (500) of manufacture for the catheter braid (100). At block (502), a first plurality of strand segments can be arranged in a first weave pattern that can have a first picks per inch. At block (504), a portion of the first portion of the plurality of strand segments can be terminated at a first transition point (210). At block (506), a second plurality of strand segments can be arranged in a second weave pattern having a second picks per inch less than the first picks per inch. The second weave pattern can be distal of the first transition point such that the second plurality of strand segments extend from a second portion of the first plurality of strand segments and such that the second weave pattern has a circumference approximately equal to the first weave pattern.

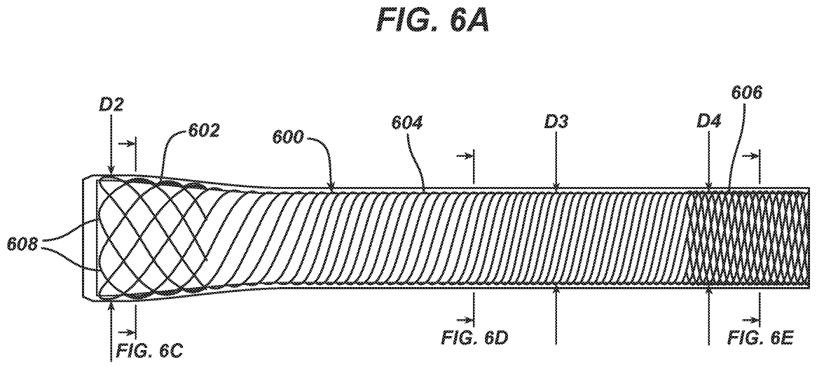

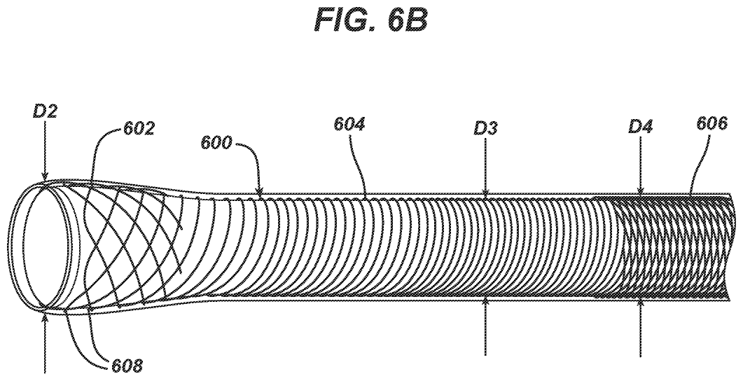

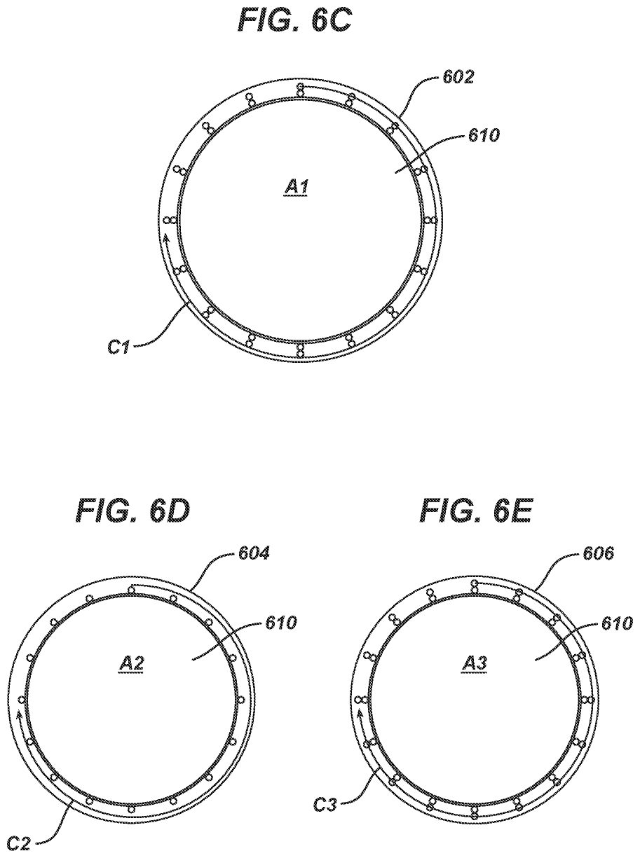

FIG. 6A is an illustration of a planar view of an example catheter having a braid (600), FIG. 6B is an isometric view of the catheter and braid (600) illustrated in FIG. 6A. FIGS. 6C, 6D, and 6E are cross-sectional views of the catheter and braid (600) as indicated in FIG. 6A.

Referring collectively to FIGS. 6A through 6E, the catheter braid (600) can include a first region (606) having a first plurality of wire segments, a second region (604) having a second plurality of wire segments fewer than the first plurality of wire segments, and a third region (602) having a third plurality of wire segments with more wire segments than the second plurality of wire segments. At least a portion of the third region (602) plurality of wire segments can include distal hoops (608). These distal hoops (608) within the third region (602) can also be described as funnel atraumatic end, purposed to minimize trauma to surrounding tissue while maintaining push efficiency. The distal hoops (608) can be formed by folding back braid strands and weaving them into the coil extending from the second region (604).

Alternatively, the distal hoops (608) can be formed by first wrapping 8 wires around a circumferential array of pins on a mandrel and then hook up the 16 free ends of the 8 wires to a braider, run the braid with picks per inch that varies as desired, then laser weld braid crossovers at the transitions from D4 to D3 and from D3 to D2, and then cut and remove the excess braid wire from D3.

The third region (602) can be configured to expand to an outer diameter (D2) that can be greater than an outer diameter (D3) of the second region (604) and the outer diameter (D4) of the first region (606) when unconstrained. The outer diameter (D3) of the second region (604) can be approximately equal to the outer diameter (D4) of the first region (606). The third region (602) can have a cross-sectional area (A1) that is greater than a cross-sectional area (A2) of the second region (604) and greater than a cross-sectional area (A3) of the first region (606). The cross-sectional area (A2) of the second region (604) can be approximately equal to the cross-sectional area (A3) of the first region (606). The third region (602) can have a circumference (C1) that is greater than a circumference (C2) of the second region (604) and greater than a circumference (C3) of the first region (606). The circumference (C2) of the second region (604) can be approximately equal to the circumference (C3) of the first region (606). The third region (602) can provide resistance to collapse under aspiration when the catheter braid (600) is advanced through a small guide catheter or vessel.

As illustrated, the first region (606) can have sixteen wire braids woven with eight clockwise wires and eight anti-clockwise wires. The first region (606) can have a first range of variable picks per inch that decreases distally. The second region (604) can have all eight clockwise wires removed, so that the second region (604) includes the remaining eight anti-clockwise wires wrapped in a coil. The coil can have a variable pitch that is increases distally. The third region (602) can be woven with sixteen wires and a third range of variable picks per inch and can have no wires removed. The third region (602) can include a distal portion of all of the eight anti-clockwise wires of the second region (604) that are folded back at the distal hoops (608) to form a weave with eight anti-clockwise wire portions and eight clockwise wire portions.

To maintain column strength of the catheter braid (600) and to prevent the wires from untangling during assembly, the terminations of wires between the first region (606), second region (604), and third regions (602) can be secured through a laser weld or adhesive bond. Additionally, to provide for improved push efficiency, the first region (606), second region (604), and third region (606) of the catheter braid (600) can include a variable braid angle within the respective region.

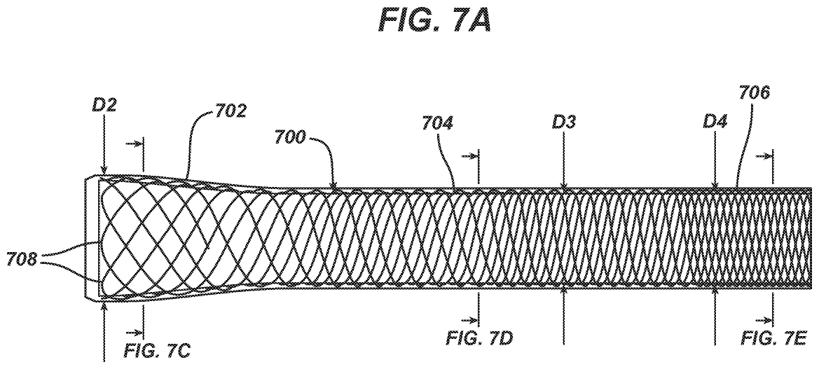

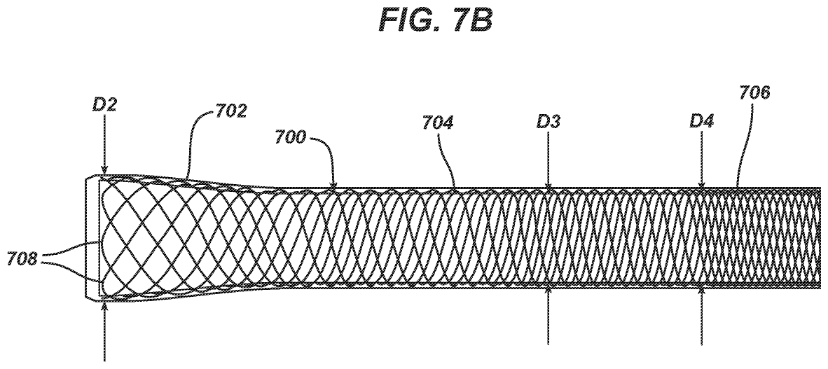

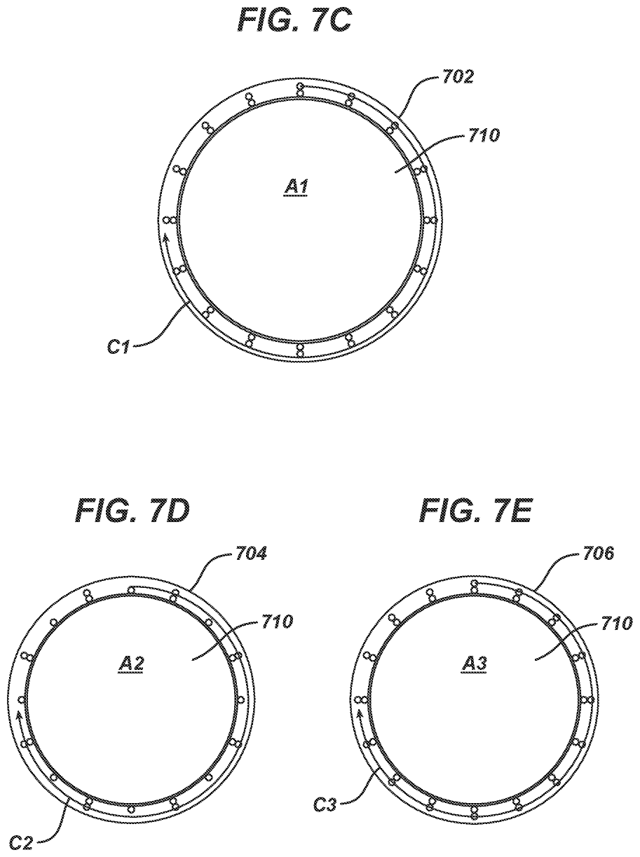

FIG. 7A is an illustration of a planar view of an example catheter having a braid (700), FIG. 7B is an isometric view of the catheter and braid (700) illustrated in FIG. 7A. FIGS. 7C, 7D, and 7E are cross-sectional views of the catheter and braid (700) as indicated in FIG. 7A. The catheter braid (700) illustrated in FIGS. 7A through 7E is similar to the braid (600) illustrated in FIGS. 6A through 6E except that where all of the wires in a given direction are removed from the second region (604) of the braid (600) in FIG. 6A, half of the clockwise wires and half of the anti-clockwise wires are removed from a second region (704) of the braid (700) in FIG. 7A.

Referring collectively to FIGS. 7A through 7E, the catheter braid (700) can include a first region (706) having a first plurality of wire segments, a second region (704) having a second plurality of wire segments fewer than the first plurality of wire segments, and a third region (702) having a third plurality of wire segments with more wire segments than the second plurality of wire segments. At least a portion of the third region (702) plurality of wire segments can include distal hoops (708). These distal hoops (708) within the third region (702) can also be described as funnel atraumatic ends, purposed to minimize trauma to surrounding tissue while maintaining push efficiency. The distal hoops (708) can be formed by folding back braid strands and weaving them into the coil extending from the second region (704).

The third region (702) can be configured to expand, when unconstrained, to an outer diameter (D2), cross-sectional area (A1), and/or circumference (C1) that can be greater than an outer diameter (D3, D4), cross-sectional area (A2, A3), and/or circumference (C2, C3) of the second region (704) and/or the first region (706). The outer diameter (D3, D4), cross-sectional area (A2, A3), and/or circumference (C2, C3) of the second region (704) can be approximately equal to that of the first region (706). The third region (702) can provide resistance to collapse under aspiration when the catheter braid (700) is advanced through a small guide catheter or vessel.

As illustrated, the first region (706) can have sixteen wire braids woven with eight clockwise wires and eight anti-clockwise wires. The first region (706) can have a first range of variable picks per inch that decreases distally. The second region (704) can have all four clockwise wires removed and four anti-clockwise wires removed, so that the picks per inch halves at the transition from the first region (706) to the second region (704). The second region (704) can have a second range of variable picks per inch that decreases distally. The third region (702) can be woven with sixteen wires and a third range of variable picks per inch with no wires removed. The third region (702) can include a distal portion of all of the eight wires of the second region (704) that are folded back at the distal hoops (708) to form a weave with eight anti-clockwise wire portions and eight clockwise wire portions.

To maintain column strength of the catheter braid (700) the terminations of wires between the first region (706), second region (704), and third regions (702) can be secured through a laser weld or adhesive bond. Additionally, to provide for improved push efficiency, the first region (706), second region (704), and third region (702) of the catheter braid (700) can include a variable braid angle within the respective region.

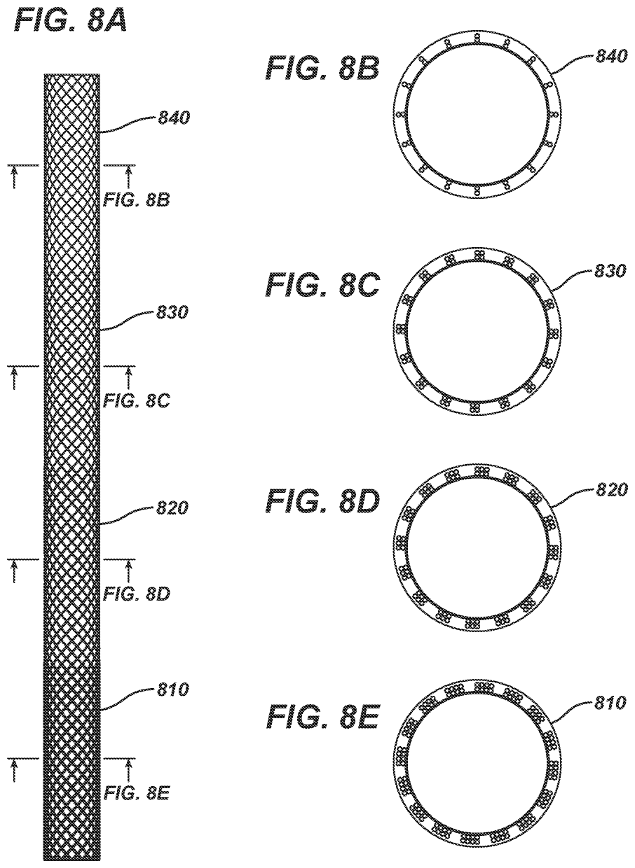

FIG. 8A is an illustration of an example embodiment of a braid 800 having a decreasingly dense weave from a first portion (810), second portion (820), third portion (830), and fourth portion (840). FIGS. 8B, 8C, 8D, and 8E are cross-sectional illustrations of the braid (800) as indicated in FIG. 8A. The first portion (810) of the braid (800) has a wires grouped in fours in a four-by-four weave. One wire in each of the groups of the first portion (810) is terminated at a junction between the first portion (810) and the second portion (820). The second portion (820) of the braid (800) has a wires grouped in threes in a three-by-three weave. One wire in each of the groups of the second portion (820) is terminated at a junction between the second portion (820) and the third portion (830). The third portion (830) of the braid (800) has a wires grouped in twos in a two-by-two weave. One wire in each of the groups of the third portion (830) is terminated at a junction between the third portion (830) and the fourth portion (840). The fourth portion (840) of the braid (800) has a wires in a one-by-one weave. The circumference, diameter, and cross-sectional area of the braid (800) is substantially consistent through the regions (810, 820, 830, 840).

The descriptions contained herein are examples of embodiments of the invention and are not intended in any way to limit the scope of the invention. As described herein, the invention contemplates many variations and modifications of a braid, including numerous wire geometries, weave patterns, and wire numbers. Further, features of the various embodiments are compatible and can be combined, resulting in variations on each embodiment. The embodiments can also be modified by techniques understood by a person skilled in the pertinent art, resulting in variations on each embodiment. These, and other modifications understood to a person skilled in the pertinent art are included in the present disclosure.

The following clauses list non-limiting embodiments of the disclosure:

Clause 1. A catheter braid comprising: a first region comprising a plurality of wire segments arranged in a first weave pattern having a first picks per inch; and a second region comprising a plurality of wire segments arranged in a second weave pattern having a second picks per inch less than the first picks per inch, the second region being distal of the first region, wherein the first weave pattern transitions to the second weave pattern at a first transitional point disposed at an intersection of the first region and the second region at which at least one wire of the plurality of wires of the first region is removed from the second region therein reducing the first picks per inch to the second picks per inch, and wherein the catheter braid comprises a lumen therethrough comprising a uniform cross section across a majority of the first region and a majority the second region.

Clause 2. The catheter braid of clause 1, wherein the first weave pattern is a 4×4 arrangement of the plurality of wire segments of the first region.

Clause 3. The catheter braid of clause 1, wherein the second weave pattern is a 4×3 arrangement of the wire segments of the second region.

Clause 4. The catheter braid of clause 1, further comprising: a third region comprising a plurality of wire segments arranged in a third weave pattern having a third picks per inch less than the second picks per inch, the third region being distal of the second region, wherein the second weave pattern transitions to the third weave pattern at a second transitional point disposed at an intersection of the second region and third region at which at least one wire of the plurality of wire of the second region is removed from the third region therein reducing the second picks per inch to the third picks per inch.

Clause 5. The catheter braid of clause 4, wherein the third weave pattern is a 3×3 arrangement of the wire segments of the third region.

Clause 6. The catheter braid of clause 4, further comprising: a fourth region comprising a plurality of wire segments arranged in a fourth weave pattern having a fourth picks per inch less than the third picks per inch, the fourth region being distal of the third region, wherein the third weave pattern transitions to the fourth weave pattern at a third transitional point disposed at an intersection of the third region and fourth region at which at least one wire of the plurality of wire of the third region is removed from the fourth region therein reducing the third picks per inch to the fourth picks per inch.

Clause 7. The catheter braid of clause 6, wherein the fourth weave pattern is a 2×2 arrangement of the wire segments of the fourth region.

Clause 8. The catheter braid of clause 6, further comprising: a fifth region comprising a plurality of wire segments arranged in a fifth weave pattern having a fifth picks per inch less than the fourth picks per inch, the fifth region being distal of the fourth region, wherein the fourth weave pattern transitions to the fifth weave pattern at a fourth transitional point disposed at an intersection of the fourth region and fifth region at which at least one wire of the plurality of wire of the fourth region is removed from the fifth region therein reducing the fourth picks per inch to the fifth picks per inch.

Clause 9. The catheter braid of clause 8, wherein the fifth weave pattern 1×1 arrangement of the wire segments of the fifth region.

Clause 10. The catheter braid of clause 1, wherein the first weave pattern comprises a braid angle approximately equal to a braid angle of the second weave pattern.

Clause 11. A catheter braid, comprising: a first region comprising a first plurality of wire segments; a second region comprising a second plurality of wire segments with fewer wire segments than the first plurality of wire segments; and a third region comprising a third plurality of wire segments with more wire segments than the second plurality of wire segments.

Clause 12. The catheter braid of clause 11, wherein at least a portion of the third plurality of wire segments comprising distal hoops.

Clause 13. The catheter braid of clause 11, wherein the third region is configured to expand to an outer diameter greater than an outer diameter of the second region and an outer diameter of the first region when unconstrained.

Clause 14. The catheter braid of clause 11, wherein the first region, second region, and/or third region comprises a variable braid angle within the respective region.

Clause 15. The catheter braid of clause 11, wherein the second plurality of wire segments consists of segments wound in a single direction.

Clause 16. The catheter braid of clause 11, wherein terminations of wires between the first, second, and third regions are secured through a laser weld or adhesive bond.

Clause 17. A catheter braid comprising: a first region comprising a first plurality of wire segments arranged in a first weave pattern, a second region comprising a second plurality of wire segments arranged in a second weave pattern, wherein the second plurality of wire segments comprises fewer wire segments than the first plurality of wire segments, and wherein the first region comprises a circumference approximately equal to a circumference of the second region.

Clause 18. The catheter braid of clause 17, wherein the first weave pattern comprises four distinct wire segments of the first plurality of wire segments traversed over four distinct wire segments of the first plurality of wire segments.

Clause 19. The catheter braid of clause 17, wherein the second weave pattern comprises four distinct wire segments of the second plurality of wire segments traversed over three distinct wire segments of the second plurality of wire segments.

Clause 20. The catheter braid of clause 17, wherein a first portion of the first plurality of wire segments terminate at a transition between the first region and the second region, and wherein the second plurality of wire segments comprise wire segments extending from a second portion of the first plurality of wire segments.

Clause 21. The catheter braid of clause 17, further comprising: a third region comprising a third plurality of wire segments arranged in a third weave pattern, wherein the third plurality of wire segments comprises fewer wire segments than the second plurality of wire segments, wherein a first portion of the second plurality of wire segments terminate at a transition between the second region and the third region, and wherein the third plurality of wire segments comprise wire segments extending from a second portion of the second plurality of wire segments.

Clause 22. The catheter braid of clause 21, wherein the third weave pattern comprises three distinct wire segments of the third plurality of wire segments traversed over three distinct wire segments of the third plurality of wire segments.

Clause 23. The catheter braid of clause 21, further comprising: a fourth region comprising a fourth plurality of wire segments arranged in a fourth weave pattern, wherein the fourth plurality of wire segments comprises fewer wire segments than the third plurality of wire segments, wherein a first portion of the third plurality of wire segments terminate at a transition between the third region and the fourth region, and wherein the fourth plurality of wire segments comprise wire segments extending from a second portion of the third plurality of wire segments.

Clause 24. The catheter braid of clause 23, wherein the fourth weave pattern comprises two distinct wire segments of the fourth plurality of wire segments traversed over two distinct wire segments of the fourth plurality of wire segments.

Clause 25. The catheter braid of clause 23, further comprising: a fifth region comprising a fifth plurality of wire segments arranged in a fifth weave pattern, wherein the fifth plurality of wire segments comprises fewer wire segments than the fourth plurality of wire segments, wherein a first portion of the fourth plurality of wire segments terminate at a transition between the fourth portion and the fifth portion, and wherein the fifth plurality of wire segments comprise wire segments extending from a second portion of the fourth plurality of wire segments.

Clause 26. The catheter braid of clause 25, wherein the fifth weave pattern comprises one wire segment of the fifth plurality of wire segments traversed over one wire segment of the fifth plurality of wire segments.

Clause 27. The catheter braid of clause 17, wherein the first weave pattern comprises a braid angle approximately equal to a braid angle of the second weave pattern.

Clause 28. A device comprising: a tubular braid comprising: a first region comprising a plurality of strand segments arranged in a first weave pattern having a first picks per inch; and a second region comprising a plurality of strand segments arranged in a second weave pattern having a second picks per inch less than the first picks per inch, the second region being distal of the first region, wherein the first weave pattern transitions to the second weave pattern at a first transitional point disposed at an intersection of the first region and the second region at which at least one strand of the plurality of strands of the first region is removed from the second region therein reducing the first picks per inch to the second picks per inch, and wherein the tubular braid comprises a uniform circumference across the first region and the second region.

Clause 29. The device of clause 28, wherein the strand segments comprise wire strands.

Clause 30. The device of clause 28, further comprising: a catheter body supported by the tubular braid.

Clause 31. The catheter braid of clause 28, wherein the first weave pattern comprises a braid angle approximately equal to a braid angle of the second weave pattern.

Clause 32. A method comprising: arranging a first plurality of strand segments in a first weave pattern having a first picks per inch; terminating a first portion of the first plurality of strand segments at a first transition point; and arranging a second plurality of strand segments in a second weave pattern having a second picks per inch less than the first picks per inch, the second weave pattern being distal of the first transition point such that the second plurality of strand segments extend from a second portion of the first plurality of strand segments and such that the second weave pattern comprises a circumference approximately equal to the first weave pattern.

What is claimed is:

1. A catheter braid comprising:
a first region comprising a plurality of wire segments arranged in a first weave pattern having a first picks per inch; and
a second region comprising a plurality of wire segments arranged in a second weave pattern having a second picks per inch less than the first picks per inch, the second region being distal of the first region,
wherein the first weave pattern transitions to the second weave pattern at a first transitional point disposed at an intersection of the first region and the second region at which at least one wire of the plurality of wire segments of the first region is removed from the second region therein reducing the first picks per inch to the second picks per inch, and
wherein the catheter braid comprises a lumen therethrough comprising a uniform cross section across a majority of the first region and a majority the second region.

2. The catheter braid of claim 1, wherein the first weave pattern is a 4×4 arrangement of the plurality of wire segments of the first region.

3. The catheter braid of claim 1, wherein the second weave pattern is a 4×3 arrangement of the wire segments of the second region.

4. The catheter braid of claim 1, further comprising:
a third region comprising a plurality of wire segments arranged in a third weave pattern having a third picks per inch less than the second picks per inch, the third region being distal of the second region,
wherein the second weave pattern transitions to the third weave pattern at a second transitional point disposed at an intersection of the second region and third region at which at least one wire of the plurality of wire segments of the second region is removed from the third region therein reducing the second picks per inch to the third picks per inch.

5. The catheter braid of claim 4, wherein the third weave pattern is a 3×3 arrangement of the wire segments of the third region.

6. The catheter braid of claim 4, further comprising:
a fourth region comprising a plurality of wire segments arranged in a fourth weave pattern having a fourth picks per inch less than the third picks per inch, the fourth region being distal of the third region,
wherein the third weave pattern transitions to the fourth weave pattern at a third transitional point disposed at an intersection of the third region and fourth region at which at least one wire of the plurality of wire segments of the third region is removed from the fourth region therein reducing the third picks per inch to the fourth picks per inch.

7. The catheter braid of claim 6, further comprising:
a fifth region comprising a plurality of wire segments arranged in a fifth weave pattern having a fifth picks per inch less than the fourth picks per inch, the fifth region being distal of the fourth region,
wherein the fourth weave pattern transitions to the fifth weave pattern at a fourth transitional point disposed at an intersection of the fourth region and fifth region at which at least one wire of the plurality of wire segments of the fourth region is removed from the fifth region therein reducing the fourth picks per inch to the fifth picks per inch.

8. The catheter braid of claim 1, wherein the first weave pattern comprises a braid angle approximately equal to a braid angle of the second weave pattern.

9. A catheter braid, comprising:
a first region comprising a first plurality of wire segments;
a second region comprising a second plurality of wire segments with fewer wire segments than the first plurality of wire segments; and
a third region comprising a third plurality of wire segments with more wire segments than the second plurality of wire segments.

10. The catheter braid of claim 9, wherein at least a portion of the third plurality of wire segments comprising distal hoops.

11. The catheter braid of claim 9, wherein the third region is configured to expand to an outer diameter greater than an outer diameter of the second region and an outer diameter of the first region when unconstrained.

12. The catheter braid of claim 9,
wherein the first region, second region, and/or third region comprises a variable braid angle within the respective region.

13. The catheter braid of claim 9, wherein the second plurality of wire segments consists of segments wound in a single direction.

14. The catheter braid of claim 9, wherein terminations of wires between the first, second, and third regions are secured through a laser weld or adhesive bond.

15. A catheter braid comprising:
a first region comprising a first plurality of wire segments arranged in a first weave pattern,
a second region comprising a second plurality of wire segments arranged in a second weave pattern,
wherein the second plurality of wire segments comprises fewer wire segments than the first plurality of wire segments, and
wherein the first region comprises a circumference approximately equal to a circumference of the second region.

16. The catheter braid of claim 15, wherein the first weave pattern comprises four distinct wire segments of the first plurality of wire segments traversed over four distinct wire segments of the first plurality of wire segments.

17. The catheter braid of claim 15, wherein the second weave pattern comprises four distinct wire segments of the second plurality of wire segments traversed over three distinct wire segments of the second plurality of wire segments.

18. The catheter braid of claim 15,
wherein a first portion of the first plurality of wire segments terminate at a transition between the first region and the second region, and
wherein the second plurality of wire segments comprise wire segments extending from a second portion of the first plurality of wire segments.

19. The catheter braid of claim 15, further comprising:
a third region comprising a third plurality of wire segments arranged in a third weave pattern, wherein the third plurality of wire segments comprises fewer wire segments than the second plurality of wire segments,
wherein a first portion of the second plurality of wire segments terminate at a transition between the second region and the third region, and
wherein the third plurality of wire segments comprise wire segments extending from a second portion of the second plurality of wire segments.

20. The catheter braid of claim 15, wherein the first weave pattern comprises a braid angle approximately equal to a braid angle of the second weave pattern.
